(19) United States
(12) Patent Application Publication
(10) Pub. No.: US 2022/0389490 A1
(43) Pub. Date: Dec. 8, 2022

(54) METHODS FOR ANALYZING AND AUTHENTICATING A SAMPLE FROM A SUBJECT

(71) Applicant: Elite Medical Laboratory Solutions LLC, Tomball, TX (US)

(72) Inventors: Saad ALSAAB, Spring, TX (US); Mehran HAIDARI, Katy, TX (US); Sravan Kumar MANSANI, Tampa, FL (US)

(21) Appl. No.: 17/741,116

(22) Filed: May 10, 2022

Related U.S. Application Data
(60) Provisional application No. 63/193,468, filed on May 26, 2021.

(51) Int. Cl. C12Q 1/6827; C12N 15/10; G01N 33/94

(52) U.S. Cl. CPC C12Q 1/6827; C12N 15/1003; G01N 33/94; C12Q 2600/156

(57) ABSTRACT

The present disclosure relates to methods of analyzing and authenticating a sample from a subject. Benefits of the methods disclosed herein can include the detection of multiple analytes in a whole blood sample, and the quantitative measurement of amounts of multiple drugs, or their metabolites, present in a single low volume whole blood sample. A benefit of the methods disclosed herein can include a combination of analyzing drugs or metabolites in a blood sample, and authenticating the blood sample, or a body sample, as being taken from the subject. Additional benefits of the methods herein can be safe, secure, accurate, and reliable authentication of blood samples and other body samples from a subject.

### CROSS-REFERENCE TO RELATED APPLICATION

This U.S. Application claims priority to U.S. Provisional No. 63/193,468, filed on May 26, 2021, the entirety of which is incorporated by reference.

### TECHNICAL FIELD

The present disclosure relates to methods of analyzing and authenticating a sample from a subject. Benefits of the methods disclosed herein can include the detection of multiple analytes in a whole blood sample, and the quantitative measurement of amounts of multiple drugs, or their metabolites, present in a single low volume whole blood sample. A benefit of the methods disclosed herein can include a combination of analyzing drugs or metabolites in a blood sample, and authenticating the blood sample, or a body sample, as being taken from the subject. Additional benefits of the methods herein can be the safe, secure, accurate, and reliable authentication of blood samples and other body samples from a subject.

### BACKGROUND

The testing of biological specimens for the presence or absence of specified drugs or their metabolites has wide applications in medical diagnostics, clinical research, drug development, employment, sports, and law enforcement. Various methods exist for the detection and measurement of analytes in blood samples. Liquid chromatography mass spectrometry (LCMS) is currently the gold standard for blood analysis, having the necessary sensitivity and specificity to measure more than 100 analytes in a single drop of blood. A dry blood spot (DBS) has been widely used to measure a limited number of analytes; however, DBS imposes inherent limitations on the accuracy of measurements. Some medical device companies have recognized these limitations and developed sampling methods and products designed to provide whole blood samples. However, there remains a need for methods that bridge the gap between such state-of-the-art blood sampling devices and LCMS analysis. There remains a need for improved accuracy and sensitivity in the analysis of multiple drugs and their metabolites in low volume whole blood samples and in other types of body samples.

The accuracy of sample analysis notwithstanding, there is always a risk that a subject can substitute their sample with another person's sample, or modify a sample, in order to “cheat” the test. This risk can mean that in order to ensure the authenticity of specimens, clinicians or other personnel have to witness the sample being collected, which can result in privacy and safety issues. There remains a need for improved methods of authenticating samples that can safely, accurately, and reliably verify that the results of the sample analysis actually correspond to the subject.

### SUMMARY

Methods of analyzing and authenticating a sample from a subject are disclosed herein. In various embodiments, the method includes: providing a blood volume of a blood sample from the subject contained within an absorbent probe of a biological fluid sampling device; forming an extracted blood sample by contacting the absorbent probe with an extraction volume of an extraction solution; forming a liquid chromatography blood sample by contacting the extracted blood sample with a liquid chromatography volume of a liquid chromatography blood sample solution; provided that a drug or metabolite analyte is present in the liquid chromatography blood sample, detecting the drug or metabolite analyte by performing liquid chromatography on the liquid chromatography blood sample and then mass spectroscopy; providing a body sample from the subject; forming purified DNA by purifying the body sample; forming a sample DNA fingerprint by performing a polymerase chain reaction (PCR) on the purified sample DNA; and authenticating the sample from the subject by comparing the sample DNA fingerprint to a reference DNA fingerprint of the subject, wherein the sample DNA fingerprint and the reference DNA fingerprint comprise genomic DNA markers unique to the subject.

In an embodiment, the body sample includes a portion of the extracted blood sample, a blood sample, a serum sample, a buccal swab sample, a saliva sample, a urine sample, a hair follicle sample, a tissue sample, or a combination thereof. In certain embodiments, the subject is a patient, a criminal suspect, a drug test subject, an athlete, a sports player, or an employee.

In certain embodiments, the blood sample has a volume of from about 20 μl to about 30 μl. In certain embodiments, the buccal swab sample has a volume of from about 100 μl to about 300 μl. In certain embodiments, the urine sample has a volume of from about 100 μl to about 300 μl. In certain embodiments, the purified sample DNA comprises from about 5 ng to about 100 ng of genomic DNA.

In certain embodiments, the method further includes: obtaining a reference body sample from the subject, wherein the reference body sample comprises a blood sample, a buccal swab sample, or a urine sample; purifying reference DNA from the baseline identification sample; and forming the reference DNA fingerprint of the subject by performing a PCR reaction on the purified reference DNA.

In certain embodiments, the method further includes reporting a genetic match or a genetic mismatch between the sample DNA fingerprint and the reference DNA fingerprint of the subject. In certain embodiments, the reference DNA fingerprint includes a database reference genetic profile. In certain embodiments, the genomic DNA markers include single nucleotide polymorphisms (SNPs), gender markers, or a combination thereof. In certain embodiments, the genomic DNA markers comprise from about 10 to about 50 SNPs or from 1 to about 5 gender markers, or a combination thereof.

In certain embodiments, the method further includes analyzing the sample DNA fingerprint by performing liquid chromatography on the purified sample DNA and then mass spectroscopy. In certain embodiments, purifying sample DNA from the body sample includes vacuum concentration of the sample DNA. In certain embodiments, provided the body sample includes cells, purifying sample DNA from the body sample includes: performing a cell lysis on the body sample to form a cell lysate; and performing an alcohol extraction on the cell lysate.

In some embodiments of methods herein, the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body, and wherein the absorbent probe includes an absorbent material. In certain embodiments, the absorbent material includes a polyolefin, polyester, polyethylene, a porous carbonized material, or a combination thereof. In certain embodiments, the absorbent probe includes an anti-coagulant.

In certain embodiments of methods herein, the blood volume is from about 5 μl to about 50 μl. In certain embodiments, the purified sample DNA has a volume of from about 2.5 μl to about 15 μl in the PCR reaction. In certain embodiments, the PCR reaction has a total volume of from about 5 μl to about 20 μl. In certain embodiments, the PCR reaction comprises from about 35 thermocycles to about 50 thermocycles.

In certain embodiments, the extraction volume is from about 50 microliters to about 200 microliters. In certain embodiments, the extraction solution includes a ratio of from about 4:1 to about 9:1 of an organic polar solvent to water. In certain embodiments, the extraction solution is an aqueous solution that includes from about 70% to about 90% volume percent of an organic polar solvent based on a total volume of the extraction solution. In certain embodiments, the organic polar solvent is selected from the group consisting of methanol, ethanol, diethylene glycol, glycerin, acetic acid, and 2-aminoethanol.

In some embodiments, the method further includes adding an internal standard volume of an internal standard solution before or during forming the extracted blood sample. In certain embodiments, the method further includes adding an internal standard volume of an internal standard solution before or during forming the liquid chromatography blood sample. In certain embodiments, the PCR reaction comprises an internal DNA quality control, an internal DNA quantity control, or a combination thereof. In certain embodiments, the internal standard volume is from about 5 microliters to about 20 microliters.

In certain embodiments, the method further includes forming the liquid chromatography blood sample by centrifuging the liquid chromatography blood sample solution for a centrifuge duration at a centrifuge rate, and then separating the liquid chromatography blood sample from any solids formed during centrifugation. In certain embodiments, the liquid chromatography blood sample solution includes an aqueous solution of from about 1:8 to about 1:2 methanol to water, and from about 0.01% to about 2% formic acid based on a total volume of the liquid chromatography blood sample solution.

In some embodiments, the method includes forming the extracted blood sample by vortexing the absorbent probe and the extraction solution for an extraction vortex duration.

In certain embodiments, the method further includes forming the liquid chromatography blood sample by removing from about 80% to 100% of a liquid from the extracted blood sample to form an extracted blood sample residue, and vortexing the extracted blood sample residue in contact with the liquid chromatography volume of the liquid chromatography blood sample solution for a residue vortex duration.

In certain embodiments, the method further includes performing liquid chromatography by pumping a first mobile phase and a second mobile phase through a solid phase column at a pressure of from about 5,000 kPa to about 35,000 kPa at a rate of from about 0.1 ml per minute to about 2 ml per minute, wherein the solid phase includes biphenyl, the first mobile phase includes from about 0.03% to about 1% formic acid and from about 0.03% to about 1% ammonium formate in water, and the second mobile phase includes from about 0.03% to about 1% formic acid in methanol.

In certain embodiments, provided that a drug or metabolite analyte is present in the liquid chromatography blood sample, the method further includes quantifying an amount of the drug or metabolite analyte in the liquid chromatography blood sample by comparing an amount of the drug or metabolite analyte detected relative to an amount of the drug or metabolite analyte in the internal standard solution.

Embodiments of a method of analyzing and authenticating a blood sample are disclosed herein. In certain embodiments, the method includes: providing a blood volume of a blood sample contained within an absorbent probe of a biological fluid sampling device, wherein the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body; forming an extracted blood sample by contacting the absorbent probe with an extraction volume of an extraction solution and an internal standard volume of an internal standard solution; forming the liquid chromatography blood sample by removing from about 80% to 100% of a liquid from the extracted blood sample to form an extracted blood sample residue, mixing the extracted blood sample residue with a liquid chromatography volume of a liquid chromatography blood sample solution, and centrifuging the liquid chromatography blood sample solution for a centrifuge duration and a centrifuge rate, and then separating the liquid chromatography blood sample from any solids formed during centrifugation; provided that a drug or metabolite analyte is present in the liquid chromatography blood sample, quantifying an amount of the drug or metabolite analyte by performing liquid chromatography on the liquid chromatography blood sample and then mass spectroscopy; providing a body sample from the subject; forming purified DNA by purifying the body sample; forming a sample DNA fingerprint by performing a polymerase chain reaction (PCR) on the purified sample DNA; comparing the sample DNA fingerprint to a reference DNA fingerprint of the subject, wherein the sample DNA fingerprint and the reference DNA fingerprint comprise genomic DNA markers unique to the subject; and determining a genetic match or a genetic mismatch between the sample DNA fingerprint and the reference DNA fingerprint of the subject based on the comparison.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 shows a flow chart of an embodiment of methods disclosed herein.

FIG. 2 shows a schematic depiction of an example of a biological fluid sampling device.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one genomic DNA marker” means one genomic DNA marker, more than one genomic DNA marker, or any combination thereof.

Unless otherwise noted, the term “about” refers to ±10% of the non-percentage number that is described, rounded to the nearest whole integer. For example, about 100 ng, would include 90 to 110 ng. Unless otherwise noted, the term “about” refers to ±5% of a percentage number. For example, about 80% would include 75 to 85%. When the term “about” is discussed in terms of a range, then the term refers to the appropriate amount less than the lower limit and more than the upper limit. For example, from about 100 μl to about 300 μl would include from 90 to 330 μl.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

The analysis of blood samples is of central importance to virtually every area of industry and research, including medical diagnostics, clinical research, drug development, employment, sports, and law enforcement. Many analytical methods of detecting and measuring the components of blood exist, with liquid chromatography mass spectrometry (LCMS) being the current gold standard. LCMS offers the necessary sensitivity and specificity for the accurate measurement of up to more than 100 analytes in a single drop of whole blood, and is widely applied to detect and measure the amounts of various drugs and their metabolites in whole blood samples. As sensitive and accurate as LCMS analysis can be, however, the quality of the results is integrally related to the quality of the collection and preparation techniques used to prepare the blood sample for LCMS analysis.

Development of methods to measure concentrations of a large number of drugs and metabolites in a drop of blood has brought about a reduction in the cost of specimen collection, as well as the prevention of injuries and other patient distress associated with regular phlebotomy blood collection. Dried blood spot (DBS) techniques have been in wide use for over 40 years. These conventional methods require collecting and drying a drop of blood on a piece of paper for later analysis of a limited number of analytes. However, the laboratory results obtained from DBS are questionable because of hematocrit error that is associated with the use of DBS. Hematocrit is a volume measurement of red blood cells in blood, expressed as a percentage. The basis of hematocrit error is that the amount of collected blood by DBS depends on the number of red blood cells in the blood sample. Blood with a high hematocrit level results in a smaller dried blood sample because the blood viscosity determines how far the blood spreads on the dried blood spotting paper, while a lower hematocrit value causes a larger dried blood sample. The properties of the paper substrate can also affect how the blood sample spreads, and the blood cells themselves can cause variations in the amount of analyte that can be extracted from the surface of the DBS card. The hematocrit level can also vary depending on other multiple factors that affect the red blood cells, including the level of body hydration and the presence of diseases affecting the hematocrit level.

Volumetric absorptive micro-sampling (VAMS) is a recently developed technique that can be used to collect blood samples for preparation and analysis by LCMS. A precise small volume of blood in the microliter range is collected by dipping an absorbent tip into a pool or drop of blood, as from a minimally invasive finger prick. The blood sample in the collection tip is then dried, extracted, and analyzed. While VAMS has similarities to the dried blood spot (DBS) methods, VAMS offers some significant advantages over DBS techniques. Analogous to DBS, the whole tip is extracted in VAMS, with additional benefits of greater sampling volume accuracy, more convenient sample collection, and a simpler process that lends itself readily to automation. VAMS has also been shown to effectively eliminate the hematocrit effect. Unlike traditional DBS methods, VAMS also permits whole blood measurements to be adjusted for serum or plasma volume.

An important difference between VAMS and DBS is the lack of hematocrit effect in the VAMS method of blood collection. For example, a large portion of the female population suffers from iron deficiency anemia. Also, a significant number of the aged male population receives testosterone therapy. As a consequence, these two populations have higher red blood cell counts (RBC) and/or higher hematocrit values. In addition, hematocrit is usually elevated in smokers. Therefore, the analysis of drug concentrations in whole blood specimens that are collected by DBS methods from patients with anemia or high hematocrit are not reliable. However, VAMS blood collection can easily provide accurate measurements for the above-mentioned populations.

While developments in VAMS technology have greatly improved blood sample collection, the preparation of whole blood specimens collected by VAMS so that they may be compatible with LCMS analysis remains elusive, creating a technology gap that, between the collection of whole blood samples and the ability to analyze the whole blood by LCMS, a difficult task. There remains a need for methods to prepare VAMS collected blood samples from recent biological fluid sampling devices that can allow for effective measurement by LCMS of the concentrations of multiple drugs from one drop of blood.

Although the measurement effectiveness that may be gained from bridging the technology gap between a VAMS biological fluid sampling device and LCMS analysis can present an important advantage, there remains the problem of uncertainty as to whether a particular sample or specimen was actually taken from the subject or person receiving a measurement test. This problem exists in a number of situations where samples are collected from subjects for testing, including healthcare, sports, employment, and law enforcement settings. Samples are routinely collected and analyzed for the presence of various drugs and metabolites. However, there a sizable risk that a subject or patient may provide a false or altered sample, or switch a sample with a sample from another person, in order to “cheat” the test.

One way to ensure sample authenticity is to collect samples from subjects in the physical presence of someone who provides a witness as to the authenticity of the samples, i.e., a clinician. However, collecting samples in this way can present not only privacy issues, but safety issues due to a risk of exposure to potentially infectious materials or agents. Safety in the collection and handling of body fluids and other biological specimens is particularly relevant in the age of global pandemics, such as COVID-19.

The development of telemedicine has greatly increased the ability of healthcare personnel to interact remotely with their patients, and has increased the safety of such interactions by avoiding the risk of exposure due to in-person contact. Another part of telemedicine involves the remote self-collecting of specimens by the subjects, and transfer of the specimens by mail or courier in specialized containers to a lab for analysis. While increasing safety, however, the risk of sample tampering or falsification by subjects also increases.

Medicine has also been evolving to provide more localized care near the patients. Many urgent care offices, doctor's offices, dentist's offices, nursing homes, sports facilities, prison medical facilities, medical collection sites, and even hospitals, do not have sophisticated on-site analytical testing equipment or capabilities. Instead, these patient-facing facilities send their samples for analysis to remote regional and national labs. These patient-facing facilities then await the report from the remote lab and use that report to inform their patient interaction. The routine acquisition of large numbers of samples from large numbers of patients creates logistical challenges to ensure that samples actually came from the right subject, in terms of deceit on the part of the subject or handling error in the supply chain.

It has been discovered that the same sample can be used for both sample analysis and subject (patient) verification. For example, one way to ensure authenticity is to collect a verified reference sample from a subject at some time point, carry out measurements on the reference sample, and store the measurement data for later comparison with test sample data. The reference sample data can include data that can uniquely identify each individual and that does not change over time, such as certain genomic DNA patterns or “DNA fingerprints”. The reference sample need only be collected and analyzed once under conditions that ensure the subject's identity, and then reference data can be electronically stored and referred to at a later time whenever needed. Test samples can be analyzed to obtain test sample data that can be compared to reference data in order to authenticate the test sample, or detect a falsification. In other words, once a remove testing facility has a subject's DNA finger print, then each sample collected by a patient facing facility can use DNA analysis of the sample, along with the test actually requested, to remotely verify that the sample actually comes from the patient.

The methods disclosed herein can not only bridge the technology gap between a VAMS biological fluid sampling device and LCMS analysis by providing a process for efficiently removing a whole blood sample from a sampling device and preparing it for LCMS. The methods disclosed herein can combine this improved process with safe, secure and accurate methods to authenticate a biological fluid sample or specimen. Such embodiments can also provide a benefit of quantitative methods that can use LCMS analysis to accurately and precisely measure the amount of one or more drugs or metabolite analytes in a blood sample contained within an absorbent probe of a biological fluid sampling device, combined with additional benefits of accurate and reliable sample authentication. Such embodiments can provide a benefit of the efficient detection and quantitative measurement of many drugs and metabolite analytes in a single drop of whole blood, while adding an effective way to ensure that drop of blood came from the intended subject.

Embodiments of Methods of Analyzing and Authenticating a Sample from a Subject

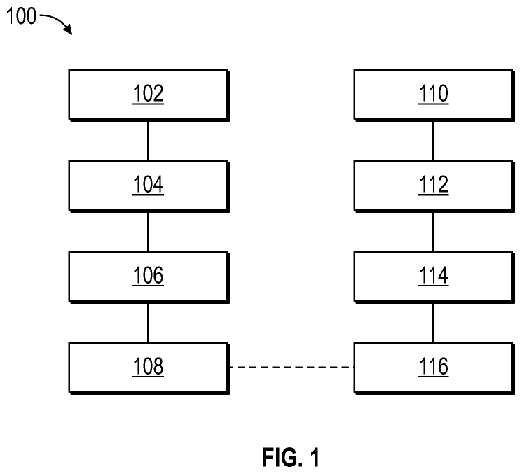

Methods of analyzing and authenticating a sample from a subject are disclosed herein. Referring to FIG. 1, as a general overview of a method disclosed herein, the method 100 includes providing a blood volume of a blood sample from the subject contained within an absorbent probe of a biological fluid sampling device 102; forming an extracted blood sample by contacting the absorbent probe with an extraction volume of an extraction solution 104; forming a liquid chromatography blood sample by contacting the extracted blood sample with a liquid chromatography volume of a liquid chromatography blood sample solution 106; detecting a drug or metabolite analyte by performing liquid chromatography on the liquid chromatography blood sample and then mass spectroscopy 108, provided that a drug or metabolite analyte is present in the liquid chromatography blood sample; providing a body sample from the subject 110; forming purified DNA by purifying the body sample 112; forming a sample DNA fingerprint by performing a polymerase chain reaction (PCR) on the purified sample DNA 114; and authenticating the sample from the subject by comparing the sample DNA fingerprint to a reference DNA fingerprint of the subject 116, wherein the sample DNA fingerprint and the reference DNA fingerprint comprise genomic DNA markers unique to the subject. In FIG. 1, the dotted line indicates that authenticating the sample 116 corresponds to authentication of the blood sample relevant to the results of drug or metabolite analyte detection 108.

Methods of analyzing and authenticating a sample from a subject are disclosed herein. In various embodiments, the method includes: providing a fluid specimen volume of a fluid specimen sample from the subject contained within an absorbent probe of a biological fluid sampling device. In an embodiment, the method includes forming an extracted specimen sample by contacting the absorbent probe with an extraction volume of an extraction solution. In an embodiment, the method includes forming a liquid chromatography specimen sample by contacting the extracted specimen sample with a liquid chromatography volume of a liquid chromatography specimen sample solution. In an embodiment, the method includes, provided that a drug or metabolite analyte is present in the liquid chromatography specimen sample, detecting the drug or metabolite analyte by performing liquid chromatography on the liquid chromatography specimen sample and then mass spectroscopy. In an embodiment, the method includes, providing a body sample from the subject. In an embodiment, the method includes forming purified DNA by purifying the body sample. In an embodiment, the method includes forming a sample DNA fingerprint by performing a polymerase chain reaction (PCR) on the purified sample DNA. In an embodiment, the method includes authenticating the sample from the subject by comparing the sample DNA fingerprint to a reference DNA fingerprint of the subject, wherein the sample DNA fingerprint and the reference DNA fingerprint comprise genomic DNA markers unique to the subject. In some embodiments, the body sample includes a portion of the fluid specimen sample. In certain embodiments, the fluid specimen sample is separate from the body sample. In certain embodiments, the fluid specimen sample includes a blood sample, a serum sample, a buccal swab sample, a saliva sample, a urine sample, or a combination thereof. In certain embodiments, the body sample includes a portion of the extracted specimen sample, a blood sample, a serum sample, a buccal swab sample, a saliva sample, a urine sample, a hair follicle sample, a tissue sample, or a combination thereof.

Embodiments herein can provide a benefit of quantitative methods that can use LCMS analysis to accurately and precisely measure the amount of one or more drugs or metabolite analytes in a fluid specimen or sample contained within an absorbent probe of a biological fluid sampling device, combined with additional benefits of accurate and reliable DNA authentication of the specimen or sample as having been taken from the subject in question.

Methods of analyzing and authenticating a sample from a subject are disclosed herein. In various embodiments, the method includes: providing a blood volume of a blood sample from the subject contained within an absorbent probe of a biological fluid sampling device; forming an extracted blood sample by contacting the absorbent probe with an extraction volume of an extraction solution; forming a liquid chromatography blood sample by contacting the extracted blood sample with a liquid chromatography volume of a liquid chromatography blood sample solution; provided that a drug or metabolite analyte is present in the liquid chromatography blood sample, detecting the drug or metabolite analyte by performing liquid chromatography on the liquid chromatography blood sample and then mass spectroscopy; providing a body sample from the subject; forming purified DNA by purifying the body sample; forming a sample DNA fingerprint by performing a polymerase chain reaction (PCR) on the purified sample DNA; and authenticating the sample from the subject by comparing the sample DNA fingerprint to a reference DNA fingerprint of the subject, wherein the sample DNA fingerprint and the reference DNA fingerprint comprise genomic DNA markers unique to the subject.

Embodiments of a method of analyzing and authenticating a blood sample are disclosed herein. In certain embodiments, the method includes: providing a blood volume of a blood sample contained within an absorbent probe of a biological fluid sampling device, wherein the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body; forming an extracted blood sample by contacting the absorbent probe with an extraction volume of an extraction solution and an internal standard volume of an internal standard solution; forming the liquid chromatography blood sample by removing from about 80% to 100% of a liquid from the extracted blood sample to form an extracted blood sample residue, mixing the extracted blood sample residue with a liquid chromatography volume of a liquid chromatography blood sample solution, and centrifuging the liquid chromatography blood sample solution for a centrifuge duration and a centrifuge rate, and then separating the liquid chromatography blood sample from any solids formed during centrifugation; provided that a drug or metabolite analyte is present in the liquid chromatography blood sample, quantifying an amount of the drug or metabolite analyte by performing liquid chromatography on the liquid chromatography blood sample and then mass spectroscopy; providing a body sample from the subject; forming purified DNA by purifying the body sample; forming a sample DNA fingerprint by performing a polymerase chain reaction (PCR) on the purified sample DNA; comparing the sample DNA fingerprint to a reference DNA fingerprint of the subject, wherein the sample DNA fingerprint and the reference DNA fingerprint comprise genomic DNA markers unique to the subject; and determining a genetic match or a genetic mismatch between the sample DNA fingerprint and the reference DNA fingerprint of the subject based on the comparison.

Embodiments of methods herein can provide a benefit of quantitative methods that can use LCMS analysis to accurately and precisely measure the amount of one or more drugs or metabolite analytes in a blood sample, including a single drop of whole blood, contained within an absorbent probe of a biological fluid sampling device. These benefits can be combined with embodiments of authenticating a sample from a subject disclosed herein, and can add additional benefits of safe, accurate and reliable sample authentication.

Embodiments of Analyzing a Sample from a Subject

Methods of analyzing and authenticating a sample from a subject are disclosed herein. Various embodiments of methods herein comprise analyzing a blood sample. In various embodiments, analyzing a blood sample includes: providing a blood volume of a blood sample from the subject contained within an absorbent probe of a biological fluid sampling device; forming an extracted blood sample by contacting the absorbent probe with an extraction volume of an extraction solution; forming a liquid chromatography blood sample by contacting the extracted blood sample with a liquid chromatography volume of a liquid chromatography blood sample solution; and provided that a drug or metabolite analyte is present in the liquid chromatography blood sample, detecting the drug or metabolite analyte by performing liquid chromatography on the liquid chromatography blood sample and then mass spectroscopy.

In certain embodiments, the blood sample includes a whole blood sample. In certain embodiments, the whole blood sample is collected from a finger prick into an absorbent probe of a biological fluid sampling device. In certain embodiments, provided that from 2 to 200 drug or metabolite analytes are present in the blood sample, from 2 to about 200 drugs or metabolite analytes present in the liquid chromatography blood sample are detected by performing liquid chromatography on the liquid chromatography blood sample and then mass spectroscopy. In certain embodiments, provided that from 2 to 150 drug or metabolite analytes are present in the blood sample, from 2 to about 150 drugs or metabolite analytes present in the liquid chromatography blood sample are detected by performing liquid chromatography on the liquid chromatography blood sample and then mass spectroscopy. In certain embodiments, provided that from 2 to 100 drug or metabolite analytes are present in the blood sample, from 2 to about 100 drugs or metabolite analytes present in the liquid chromatography blood sample are detected by performing liquid chromatography on the liquid chromatography blood sample and then mass spectroscopy. In certain embodiments, provided that from 2 to 50 drug or metabolite analytes are present in the blood sample, from 2 to about 50 drugs or metabolite analytes present in the liquid chromatography blood sample are detected by performing liquid chromatography on the liquid chromatography blood sample and then mass spectroscopy. In certain embodiments, one or more drugs or metabolite analytes present in the liquid chromatography blood sample can be detected by liquid chromatography and then mass spectroscopy on the liquid chromatography blood sample, when the one or more drugs or metabolite analytes are present at or above a ng/mL cutoff concentration.

In some embodiments of methods herein, the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body, and wherein the absorbent probe includes an absorbent material. In certain embodiments, the absorbent material includes a polyolefin, polyester, polyethylene, a porous carbonized material, or a combination thereof. In certain embodiments, the absorbent probe includes an anti-coagulant.

In certain embodiments of methods herein, the blood volume is from about 5 μl to about 50 μl. When the blood volume falls below about 5 microliters, then the method is not sensitive enough and produces false negative results. When the blood volume falls above about 50 microliters, then the method tends to produce unreliable results. In an aspect, the blood volume is from about 10 microliters to about 40 microliters. In an aspect, the blood volume is from about 20 microliters to about 30 microliters. In certain embodiments, the blood sample is dried before further treatment.

In certain embodiments, the extraction volume is from about 50 microliters to about 200 microliters. When the extraction volume falls below about 50 microliters, then the extraction of the drugs being measured is not complete, resulting in erroneously low measurements. When the extraction volume goes above about 200 microliters, then produces accurate measurements, but the process wastes solvent and requires higher drying times. In an aspect, the extraction volume is from about 80 microliters to about 170 microliters. In an aspect, the extraction volume is from about 110 microliters to about 140 microliters.

In certain embodiments, the extraction solution includes a ratio of from about 4:1 to about 9:1 of an organic polar solvent to water. In certain embodiments, the extraction solution is an aqueous solution that includes from about 70% to about 90% volume percent of an organic polar solvent based on a total volume of the extraction solution. In certain embodiments, the organic polar solvent is selected from the group consisting of methanol, ethanol, diethylene glycol, glycerin, acetic acid, and 2-aminoethanol. When the extraction solution has a ratio above or below the range of about 4:1 to about 9:1 of an organic polar solvent to water, then the ability of the process to precipitate proteins from solution before liquid chromatography is adversely affected. In an aspect, the extraction solution includes a ratio of about 5:1 to about 8:1 of an organic polar solvent to water. In an aspect, the extraction solution includes a ratio of about 6:1 to about 7:1 of an organic polar solvent to water. In certain embodiments, the extraction solution is an aqueous solution that includes from about 70% to about 90% volume percent of an organic polar solvent based on a total volume of extraction solution. In an aspect, the extraction solution is an aqueous solution that includes from about 73% to about 87% volume percent of an organic polar solvent based on a total volume of extraction solution. In an aspect, the extraction solution is an aqueous solution that includes from about 75% to about 85% volume percent of an organic polar solvent based on a total volume of extraction solution. In certain embodiments, the organic polar solvent includes methanol, ethanol, diethylene glycol, glycerin, acetic acid, or 2-aminoethanol.

In some embodiments, the method further includes adding an internal standard volume of an internal standard solution before or during forming the extracted blood sample. In certain embodiments, the method further includes adding an internal standard volume of an internal standard solution before or during forming the liquid chromatography blood sample. In certain embodiments, the internal standard volume can be about 5 microliters to about 20 microliters. In an aspect, the internal standard volume can be about 8 microliters to about 17 microliters. In an aspect, the internal standard volume can be about 10 microliters to about 15 microliters. In some embodiments, methods include adding an internal standard volume of an internal standard solution before forming the liquid chromatography blood sample. In some embodiments, methods include adding an internal standard volume of an internal standard solution during forming the liquid chromatography blood sample. Certain embodiments include, provided that a drug or metabolite analyte is present in the liquid chromatography blood sample, quantifying an amount of the drug or metabolite analyte in the liquid chromatography blood sample by comparing an amount of drug or metabolite analyte detected relative to an amount of the drug or metabolite analyte in the internal standard solution. In certain embodiments, one or more drugs or metabolite analytes present in the liquid chromatography blood sample can be quantified by liquid chromatography and then mass spectroscopy on the liquid chromatography blood sample, when the one or more drugs or metabolite analytes are present at or above a ng/mL cutoff concentration.

In certain embodiments, the method further includes forming the liquid chromatography blood sample by centrifuging the liquid chromatography blood sample solution for a centrifuge duration at a centrifuge rate, and then separating the liquid chromatography blood sample from any solids formed during centrifugation. In certain embodiments, the method further includes forming the liquid chromatography blood sample by removing from about 80% to 100% of a liquid from the extracted blood sample to form an extracted blood sample residue, and vortexing the extracted blood sample residue in contact with the liquid chromatography volume of the liquid chromatography blood sample solution for a residue vortex duration. In an aspect, the extracted blood sample residue is formed by removing from about 85% to 100% of a liquid from the extracted blood sample; in another aspect, the extracted blood sample residue is formed by removing from about 90% to 100% of a liquid from the extracted blood sample. In some embodiments, the method includes forming the extracted blood sample by vortexing the absorbent probe and the extraction solution for an extraction vortex duration.

In certain embodiments, the liquid chromatography blood sample solution includes an aqueous solution of from about 1:8 to about 1:2 methanol to water, and from about 0.01% to about 2% formic acid based on a total volume of the liquid chromatography blood sample solution. When the range of methanol falls above or below to water about 1:8 to about 1:2 methanol to water, then the ability to resolve or separate the drugs before mass specification analysis is adversely impacted. In an aspect, the liquid chromatography blood sample solution includes an aqueous solution of about 1:7 to about 1:3 methanol to water, and about 0.01 to about 1.5% formic acid based on a total volume of the liquid chromatography blood sample solution. In an aspect, the liquid chromatography blood sample solution includes an aqueous solution of about 1:6 to about 1:4 methanol to water, and about 0.01 to about 1.0% formic acid based on a total volume of the liquid chromatography blood sample solution.

In certain embodiments, the method further includes performing liquid chromatography by pumping a first mobile phase and a second mobile phase through a solid phase column at a pressure of from about 5,000 kPa to about 35,000 kPa at a rate of from about 0.1 ml per minute to about 2 ml per minute. In an aspect, the first mobile phase and the second mobile phase are pumped through a solid phase column at a pressure of from about 10,000 kPa to about 30,000 kPa. In an aspect, the first mobile phase and the second mobile phase are pumped through a solid phase column at a pressure of from about 15,000 kPa to about 25,000 kPa. In an aspect, the pumping rate is from about 0.5 ml per minute to about 1.6 ml per minute. In another aspect, the pumping rate is from about 0.9 ml per minute to about 1.2 ml per minute. In certain embodiments, the solid phase includes biphenyl, the first mobile phase includes from about 0.03% to about 1% formic acid and from about 0.03% to about 1% ammonium formate in water, and the second mobile phase includes from about 0.03% to about 1% formic acid in methanol. In an aspect, the solid phase includes biphenyl, the first mobile phase includes from about 0.1% to about 0.8% formic acid and from about 0.1% to about 0.8% ammonium formate in water, and the second mobile phase includes from about 0.1% to about 0.8% formic acid in methanol. In another aspect, the solid phase includes biphenyl, the first mobile phase includes from about 0.3% to about 0.6% formic acid and from about 0.3% to about 0.6% ammonium formate in water, and the second mobile phase includes from about 0.3% to about 0.6% formic acid in methanol.

In certain embodiments, the method includes: providing a blood volume of a blood sample contained within an absorbent probe of a biological fluid sampling device, wherein the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body; forming an extracted blood sample by contacting the absorbent probe with an extraction volume of an extraction solution and an internal standard volume of an internal standard solution; forming the liquid chromatography blood sample by removing from about 80% to 100% of a liquid from the extracted blood sample to form an extracted blood sample residue, mixing the extracted blood sample residue with a liquid chromatography volume of a liquid chromatography blood sample solution, and centrifuging the liquid chromatography blood sample solution for a centrifuge duration and a centrifuge rate, and then separating the liquid chromatography blood sample from any solids formed during centrifugation; provided that a drug or metabolite analyte is present in the liquid chromatography blood sample, quantifying an amount of the drug or metabolite analyte by performing liquid chromatography on the liquid chromatography blood sample and then mass spectroscopy.

Biological Fluid Sampling Devices of Various Embodiments

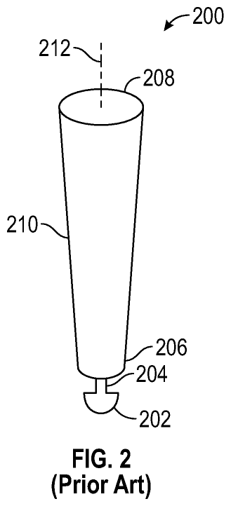

Various methods embodied herein include providing a blood volume of a blood sample contained within an absorbent probe of a biological fluid sample device. As shown in FIG. 2, an embodiment of a biological fluid sampling device 200 can include an absorbent probe 202 mounted to a post 204 of biological fluid sampling device 200, having smaller diameter first end 206, larger diameter second end 208, and elongated and tapered body 210 extending along a longitudinal axis 212. In such embodiments, the biological fluid sample device includes a VAMS device that is useful for the collection of precise low blood volumes in microliter amounts. Such a blood volume can in some embodiments be collected from a finger prick into the absorbent probe of the biological fluid sampling device. In certain embodiments, the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body, and wherein the absorbent probe includes an absorbent material.

In certain embodiments, the absorbent material includes a porous, hydrophilic polymeric material. Such a hydrophilic polymeric material may be initially hydrophilic, or the surfaces of the material may be converted into a hydrophilic state by one or more treatments. Such treatments may include an adsorptive treatment with surfactants, treatment with plasma, covalent modification, or other suitable treatment. In certain embodiments, the absorbent material includes a polyolefin, polyester, polyethylene, a porous carbonized material, or a combination thereof. In various embodiments, the absorbent material is sufficiently porous in order to absorb fluid. In certain embodiments, the internal pore volume of the absorbent material is between about 30% and 50% of the total volume of the material. In certain embodiments, the absorbent material contains pores of about 20-50 microns in diameter. In certain embodiments, the absorbent probe includes an anti-coagulant. In certain embodiments wherein the absorbent material includes a polyolefin, the polyolefin can include a hydrophilic polyolefin having a density of from about 0.1 to 1 g/cc. In certain embodiments wherein the absorbent material includes a polyethylene, the polyethylene can include a hydrophobic polyethylene having a non-porous density of about 0.6 g/cc that is plasma treated to make it hydrophilic.

In various embodiments, the absorbent probe is contacted directly with a biological fluid, such as blood. In certain embodiments, the absorbent probe is contacted directly with the blood of a human or animal while the blood is on the human or animal. In other embodiments, the blood may be transferred from the human or animal to a container, and the absorbent probe is contacted with the blood in the container. In certain embodiments, the absorbent probe has a cylindrical, conical, circular, square or triangular shape that is suitable for use with pipette holders or sample tubes. In certain embodiments, the absorbent probe is detachably or reversibly mounted to a holder or a post extending from the holder. In certain embodiments, the absorbent probe can be detached or removed from the holder or post and placed into a sample tube or other suitable container. In certain embodiments, the blood sample contained within the absorbent probe is dried before preparation of the blood sample for analysis. The blood sample may be covered by a suitable protective sheath or placed in a sealed container so that the blood sample can be transported to another location for analysis. In certain embodiments, an extracted blood sample is formed by providing a blood volume of a blood sample contained within an absorbent probe of a biological fluid sampling device, wherein the absorbent probe is detached from a holder and placed in a suitable sample container, and contacting the absorbent probe with an extraction volume of an extraction solution in the sample container.

Embodiments of analyzing a sample from a subject herein can provide a benefit of the accurate analysis of one or more drugs or metabolite analytes in a sample containing a small fluid volume. Such embodiments can provide a benefit of the efficient detection and quantitative measurement of many drugs and metabolite analytes in a single drop of whole blood. Such embodiments can also provide benefits of safety and convenience, in that they allow the collection of samples at a remote location into sealed or other suitable containers, so that they can then be easily transported to another location, such as a lab, for analysis.

Embodiments of Authenticating a Sample from a Subject

Embodiments of methods of analyzing and authenticating a sample from a subject are disclosed herein. In various embodiments, the methods include authenticating a sample from a subject or patient. In various embodiments, authenticating a sample from a subject includes: providing a body sample from the subject; forming purified DNA by purifying the body sample; forming a sample DNA fingerprint by performing a polymerase chain reaction (PCR) on the purified sample DNA; and authenticating the sample from the subject by comparing the sample DNA fingerprint to a reference DNA fingerprint of the subject, wherein the sample DNA fingerprint and the reference DNA fingerprint comprise genomic DNA markers unique or particular to the subject.

It is understood that DNA markers are generally unique or indicative of a subject or patient. However, there can be exceptions such as clones or genetic twins. In an embodiment, the DNA markers are unique or rare or indicative of the patient such that the odds of an incorrect match are more than 100,000 to 1.

In an embodiment, the body sample includes a portion of the blood sample, or a portion of the extracted blood sample that is analyzed to detect a drug or metabolite analyte. In such embodiments, the portion is used to provide purified DNA and to form a sample DNA fingerprint; thus, the sample DNA fingerprint that is compared to a reference DNA fingerprint is derived directly from the blood sample from the subject. Such embodiments can provide a benefit of a direct authentication of the sample from the subject. Such embodiments can also provide a benefit of the need to collect only a single sample from a subject, because the single sample provides both the blood sample for the analysis, and the body sample for the authentication.

In certain embodiments, the body sample can include a sample that is different from the blood sample from the subject. Situations in which such embodiments can occur is when the fluid specimen or blood sample does not contain a sufficient volume for both the analysis and the authentication, or otherwise the DNA fingerprint cannot be successfully obtained from a portion of the blood sample or extracted blood sample. In certain embodiments, the body sample can include a blood sample, a serum sample, a buccal swab sample, a saliva sample, a urine sample, a hair follicle sample, a tissue sample, or a combination thereof. In certain embodiments, the body sample can be collected from the subject at the same or a different time or place as the blood sample. In certain embodiments, the blood sample from the subject can include a portion of the body sample. Such embodiments can provide a benefit of greater versatility to embodiments of the methods herein, for example, in situations where the original blood sample is lost, or becomes damaged or degraded.

Embodiments of authenticating a sample from a subject can provide a benefit of applicability to a wide variety of situations where samples are routinely collected from subjects and analyzed for one or more drugs or metabolites. Such embodiments can be advantageous for such uses in healthcare, employment, drug testing, sports, and law enforcement settings. In certain embodiments, the subject is a patient, a criminal suspect, a drug test subject, an athlete, a sports player, or an employee.

In certain embodiments of a body sample herein, the blood sample has a volume of from about 20 μl to about 30 μl. In certain embodiments, the blood sample has a volume of from about 22 μl to about 28 μl. In certain embodiments, the blood sample has a volume of from about 23 μl to about 25 μl. In certain embodiments, the buccal swab sample has a volume of from about 100 μl to about 300 μl. In certain embodiments, the buccal swab sample has a volume of from about 150 μl to about 250 μl. In certain embodiments, the buccal swab sample has a volume of from about 175 μl to about 200 μl. In certain embodiments, the urine sample has a volume of from about 100 μl to about 300 μl. In certain embodiments, the urine sample has a volume of from about 150 μl to about 250 μl. In certain embodiments, the urine sample has a volume of from about 175 μl to about 200 μl. In certain embodiments, the purified sample DNA comprises from about 5 ng to about 100 ng of genomic DNA. In certain embodiments, the purified sample DNA comprises from about 10 ng to about 80 ng of genomic DNA. In certain embodiments, the purified sample DNA comprises from about 30 ng to about 50 ng of genomic DNA.

In certain embodiments, the method further includes: obtaining a reference body sample from the subject, wherein the reference body sample comprises a blood sample, a buccal swab sample, or a urine sample; purifying reference DNA from the baseline identification sample; and forming the reference DNA fingerprint of the subject by performing a PCR reaction on the purified reference DNA.

In certain embodiments, the method further includes reporting a genetic match or a genetic mismatch between the sample DNA fingerprint and the reference DNA fingerprint of the subject. In certain embodiments, the reference DNA fingerprint includes a database reference genetic profile. In certain embodiments, the genomic DNA markers include single nucleotide polymorphisms (SNPs), gender markers, or a combination thereof. In certain embodiments, the genomic DNA markers comprise from about 10 to about 50 SNPs, or from 1 to about 5 gender markers, or a combination thereof. In certain embodiments, the genomic DNA markers comprise from about 20 to about 40 SNPs. In certain embodiments, the genomic DNA markers comprise from about 30 to about 40 SNPs. In certain embodiments, the genomic DNA markers comprise from 2 to about 4 gender markers.

In certain embodiments, the method further includes analyzing the sample DNA fingerprint by performing liquid chromatography on the purified sample DNA and then mass spectroscopy. In certain embodiments, purifying sample DNA from the body sample includes vacuum concentration of the sample DNA. In certain embodiments, provided the body sample includes cells, purifying sample DNA from the body sample includes: performing a cell lysis on the body sample to form a cell lysate; and performing an alcohol extraction on the cell lysate.

In certain embodiments, the purified sample DNA has a volume of from about 2.5 μl to about 15 μl in the PCR reaction. In certain embodiments, the purified sample DNA has a volume of from about 3.0 μl to about 10 μl in the PCR reaction. In certain embodiments, the purified sample DNA has a volume of from about 5.0 μl to about 8 μl in the PCR reaction. In certain embodiments, the PCR reaction has a total volume of from about 5 μl to about 20 μl. In certain embodiments, the PCR reaction has a total volume of from about 8 μl to about 15 μl. In certain embodiments, the PCR reaction has a total volume of from about 10 μl to about 12 μl. In certain embodiments, the PCR reaction comprises from about 35 thermocycles to about 50 thermocycles. In certain embodiments, the PCR reaction comprises from about 38 thermocycles to about 48 thermocycles. In certain embodiments, the PCR reaction comprises from about 40 thermocycles to about 45 thermocycles.

In certain embodiments, the PCR reaction comprises an internal DNA quality control, an internal DNA quantity control, or a combination thereof. In certain embodiments, the internal standard volume is from about 5 microliters to about 20 microliters. In certain embodiments, the internal standard volume is from about 8 microliters to about 15 microliters. In certain embodiments, the internal standard volume is from about 10 microliters to about 12 microliters.

### EXAMPLES

### Example 1

### Urine Sample Analysis

Sample preparation procedure for urine (urine drug testing)
a. Allow the working solutions to come to room temperature. Make sure that all the solutions are completely thawed and are at the room temperature.
b. Turn on the incubator, and set it to 55° C.
c. Label a complete set of 1.5 mL micro-centrifuge tubes that correspond to each of the intended samples for the batch.
d. Add 40 μL of the IMCS buffer to all of the micro-centrifuge tubes.
e. Add 30 μL of β-glucuronidase enzyme to all of the micro-centrifuge tubes.
f. Add 10 μL of Internal Standard (IS) stock solution to all of the micro-centrifuge tubes, except the Double Blank sample. (Instead of IS, add 10 μL of methanol to the Double Blank sample).
g. Add 100 μL of each unknown (patient urine samples) to the respectively labelled micro-centrifuge tubes. (Note: the micro-centrifuge tubes will herein be referred to as samples).
h. Vortex all samples for 10 seconds.
i. Incubate all samples at 55° C. for 30 minutes.
j. Add 500 μL of Sample Diluent to all samples.
k. Vortex all samples for 10 seconds.
l. Centrifuge all samples at 3,700 rpm for 15 minutes.
m. Transfer as much of the supernatant of each sample as possible (Approx. 500-600μL) into LCMS vials labelled identically to the micro-centrifuge tubes of the corresponding samples.
   Note: The volume need not be exact because the concentrations of the Analytes and Internal Standards are already set.
n. Place vials in the LCMS autosampler. Samples are now ready to be analyzed on LCMS.

### Urine Sample DNA Preparation (Identity Test) Procedure

1. In a 15 ml conical tube, add 10 ml of patient urine sample and centrifuge at 3500 RPM for 15 minutes. Discard the supernatant in the sink and keep whatever is left over in the bottom of the 15 ml tube.

2. Pipet 20 μl Protease (or proteinase K) into the bottom of a 1.5 ml microcentrifuge tube.

3. Add 200 μl urine precipitant sample from step 1 to the microcentrifuge tube. If the sample volume is less than 200 μl, add the appropriate volume of Phosphate Buffered Saline (PBS).

4. Add 200 μl Buffer AL to the sample. Mix by pulse-vortexing for 15 seconds.

5. Incubate at 56° C. for 10 minutes.

6. Briefly centrifuge the 1.5 ml microcentrifuge tube to remove drops from the inside of the lid.

7. Add 200 μl ethanol (96-100%) to the sample, and mix again by pulse-vortexing for 15 seconds. After mixing, briefly centrifuge the 1.5 ml microcentrifuge tube to remove drops from the inside of the lid.

8. Carefully apply the mixture from step 6 to the Mini spin column (in a 2 ml collection tube) without wetting the rim. Close the cap, and centrifuge at 6000×g (8000 rpm) for 1 minute. Place the Mini spin column in a clean 2 ml collection tube (provided), and discard the tube containing the filtrate.

9. Carefully open the Mini spin column and add 500 μl Buffer AW1 without wetting the rim. Close the cap and centrifuge at 6000×g (8000 rpm) for 1 minute. Place the Mini spin column in a clean 2 ml collection tube (provided), and discard the collection tube containing the filtrate.

10. Carefully open the Mini spin column and add 500 μl Buffer AW2 without wetting the rim. Close the cap and centrifuge at full speed (20,000×g; 14,000 rpm) for 3 minutes.

11. Place the Mini spin column in a new 2 ml collection tube and discard the old collection tube with the filtrate. Centrifuge at full speed for 1 min.

12. Place the Mini spin column in a clean 1.5 ml microcentrifuge tube and discard the collection tube containing the filtrate. Carefully open the Mini spin column and add 200 μl Buffer AE or distilled water.
Incubate at room temperature (15-25° C.) for 1 minute, and then centrifuge at 6000×g (8000 rpm) for 1 minute.
Incubating the Mini spin column loaded with Buffer AE or water for 5 minutes at room temperature before centrifugation generally increases DNA yield.

### Example 2

### Blood Sample Analysis

### Blood Collection

Blood was collected using an FDA-approved MITRA® device (NEOTERYX®, Torrance, Calif. 90501). The MITRA® sampling device uses a Volumetric Absorptive Micro sampling (VAMS®) novel sampling technique that allows the straightforward collection of an accurate volume of blood (approximately 30 microliters) from a drop of blood by dipping an absorbent polymeric tip into it. The res tin blood microsample was dried for more than three hours and analyzed as a whole.

### Sample Preparation Protocol

A volume of 120 microliters of methanol: water (80:20) extraction solvent was added to a 2 mL microcentrifuge tube, followed by 10 microliters of internal standard stock that contained an internal standard for every analyte. The Volumetric Absorptive Micro sampling (VAMS®) tip was separated from the handler and transferred into the microcentrifuge tube. The tube was vortexed for 30 mins at speed 6 on a vortex mixer. The tip was removed from the tube, and the liquid portion in the tube was dried under a gentle stream of nitrogen gas at 15 psi and 37° C. for 10 mins, on a nitrogen drying unit. After the nitrogen drying process, a volume of 25 microliters of methanol and 75 microliters of 0.1% Formic Acid in water was added. The tube was vortexed for 30 minutes at speed 6 on a vortex mixer. The tubes were centrifuged for 15 minutes. The supernatant was transferred from the tubes into a Liquid Chromatography Mass Spectrometry (LCMS) vial and submitted to LCMS analysis.

### Analytical Method

A dilute and shoot confirmatory LCMS method measured 85 drugs and metabolites; these included major categories of illicit drugs and drugs commonly prescribed for chronic pain patients. Table 1 shows the list of drugs/metabolites measured and their cutoff concentrations.

LC—MS-MS analysis was performed on a Shimadzu Nexera XR high-pressure liquid chromatography (HPLC) system (SHIMADZU® Corporation, Kyoto, Japan) coupled with a SCIEX® 4500 mass spectrometer (AB SCIEX®, Framingham, Mass.).

The experiments were carried out on 85 drugs and metabolites (Table 1), of which 83 were detected in a positive ionization method and 2 in a negative ionization method (butalbital and phenobarbital). In both methods, the chromatography separation was performed on a Raptor™ Biphenyl column, 2.7 μm, 50×3.0 mm (RESTEK®, Bellefonte, Pa.) using gradient elution containing 0.1% formic acid and 0.1% ammonium formate in water (mobile phase A) and 0.1% formic acid in methanol (mobile phase B). A Raptor™ Biphenyl EXP Guard Column Cartridge (2.7 μm, 5×3.0 mm) was installed preceding the bi-phenyl analytical column for cleaning up the samples.

Analytes were detected by mass spectrometry using scheduled multiple reaction monitoring (MRM) in either positive or negative electrospray ionization (ESI) modes. Two characteristic MRM transitions were monitored for each analyte. The MRM ratios, which are defined as the peak area ratios between primary and secondary ion transitions, were only acceptable if within <30% for all analytes.

The data were collected using the AB SCIEX® Analyst, 1.7 software and quantified with the MultiQuant, 2.1 software. Urine drug screening tests (immunoassay) were performed using a BECKMAN® AU 680 chemistry autoanalyzer (BECKMAN COULTER®, Brea, Calif.).

TABLE 1

| | Cut Off |
|---|---|
| Analyte | (ng/mL) |
| 6-MAM | 10 |
| 7-Aminoclonazepam | 50 |
| Acetaminophen | 250 |
| a-Hydroxyalprazolam | 25 |
| a-Hydroxymidazolam | 25 |
| Alprazolam | 25 |
| Amitriptyline | 25 |
| Amphetamine | 100 |
| Aripiprazole | 50 |
| Atomoxetine | 50 |
| Baclofen | 50 |
| Benzoylecgonine | 50 |
| Buprenorphine | 10 |
| Bupropion | 10 |
| Buspirone | 10 |
| Butalbital | 100 |
| Carbamazepine | 50 |
| Carisoprodol | 50 |
| Citalopram | 50 |
| Clozapine | 50 |
| Codeine | 50 |
| Cotinine | 100 |
| Cyclobenzaprine | 25 |
| Desipramine | 25 |
| Desmethyltapentadol | 50 |
| Desmethyltramadol | 100 |
| Desmethylvenlafaxine | 100 |
| Dextromethorphan | 100 |
| Dextrorphan | 100 |
| Doxepin | 25 |
| Duloxetine | 25 |
| EDDP | 100 |
| Fentanyl | 2.5 |
| Flunitrazepam | 50 |
| Fluoxetine | 50 |
| Gabapentin | 1000 |
| Haloperidol | 50 |
| Hydrocodone | 50 |
| Hydromorphone | 50 |
| Hydroxytriazolam | 25 |
| Ibuprofen | 500 |
| Ketamine | 25 |
| Levetiracetam | 50 |
| Lorazepam | 50 |
| MDA | 100 |
| MDMA | 100 |
| Meprobamate | 100 |
| Methadone | 100 |
| Methamphetamine | 100 |
| Morphine | 50 |
| Naloxone | 10 |
| Naltrexone | 50 |
| Naproxen | 100 |
| Norbuprenorphine | 25 |
| Nordiazepam | 50 |
| Norfentanyl | 5 |
| Norhydrocodone | 50 |
| Norketamine | 25 |
| Noroxycodone | 50 |
| Norpropoxyphene | 50 |
| Nortriptyline | 25 |
| Olanzapine | 50 |
| Oxazepam | 50 |
| Oxycodone | 50 |
| Oxymorphone | 50 |
| Paroxetine | 50 |
| Phencyclidine | 25 |
| Phenobarbital | 100 |
| Phentermine | 100 |
| Pregabalin | 200 |
| Propoxyphene | 50 |
| Protriptyline | 50 |
| Pseudoephedrine | 25 |
| Quetiapine | 50 |
| Risperidone | 25 |
| Salicylic Acid | 500 |
| Sertraline | 50 |
| Tapentadol | 50 |
| Temazepam | 50 |
| THCA | 15 |
| Topiramate | 50 |
| Tramadol | 100 |
| Venlafaxine | 100 |
| Zaleplon | 10 |
| Zolpidem | 10 |

### Example 3

### DNA Purification

Protocol: DNA Purification from Dried Blood Spots (QIAamp® DNA Mini Kit, QIAgen).

The following protocol was used for purification of total (genomic, mitochondrial, and viral) DNA from blood, both untreated and treated with anticoagulants, which has been spotted and dried on filter paper (Schleicher and Schuell 903).

1. Separate the Volumetric Absorptive Micro sampling (VAMS®) tip from the handler and transfer into the microcentrifuge tube.

2. Add 20 μl proteinase K stock solution. Mix by vortexing, and incubate at 56° C. for 1 h. Briefly centrifuge to remove drops from inside the lid.

3. Add 200 μl Buffer AL to the sample. Mix thoroughly by vortexing, and incubate at 70° C. for 10 min. Briefly centrifuge to remove drops from inside the lid. To ensure efficient lysis, the sample and Buffer AL are mixed immediately and thoroughly. Note: Do not add proteinase K directly to Buffer AL.

A white precipitate may form when Buffer AL is added to the sample. In most cases, the precipitate will dissolve during incubation. The precipitate does not interfere with the QIAamp procedure or with any subsequent application.

4. Add 200 μl ethanol (96-100%) to the sample, and mix thoroughly by vortexing. Briefly centrifuge to remove drops from inside the lid.

5. Carefully apply the mixture from step 5 to the QIAamp Mini spin column (in a 2 ml collection tube) without wetting the rim. Close the cap, and centrifuge at 6000×g (8000 rpm) for 1 min. Place the QIAamp Mini spin column in a clean 2 ml collection tube (provided), and discard the tube containing the filtrate. Close each QIAamp Mini spin column to avoid aerosol formation during centrifugation.

6. Carefully open the QIAamp Mini spin column and add 500 μl Buffer AW1 without wetting the rim. Close the cap and centrifuge at 6000×g (8000 rpm) for 1 min. Place the QIAamp Mini spin column in a clean 2 ml collection tube (provided), and discard the collection tube containing the filtrate.

7. Carefully open the QIAamp Mini spin column and add 500 μl Buffer AW2 without wetting the rim. Close the cap and centrifuge at full speed (20,000×g; 14,000 rpm) for 3 min.

8. Recommended: Place the QIAamp Mini spin column in a new 2 ml collection tube (not provided) and discard the old collection tube with the filtrate. Centrifuge at full speed for 1 min. This step helps to eliminate the chance of possible Buffer AW2 carryover.

9. Place the QIAamp Mini spin column in a clean 1.5 ml microcentrifuge tube (not provided), and discard the collection tube containing the filtrate. Carefully open the QIAamp Mini spin column and add 150 μl Buffer AE or distilled water. Incubate at room temperature (15-25° C.) for 1 min, and then centrifuge at 6000×g (8000 rpm) for 1 min.

Three punched-out circles (3 mm diameter) typically yield 150 ng and 75 ng of DNA from anticoagulated and untreated blood, respectively. If the yield from untreated blood is not sufficient, use 6 circles per prep instead of 3.

The volume of the DNA eluate used in a PCR assay should not exceed 10%; for example, for a 50 μl PCR, add no more than 5μl of eluate.

### Example 4

### DNA Fingerprint Analysis

DNA fingerprint analysis was performed on the genomic DNA samples using the iPLEX® Pro Sample Integrity Panel, which analyzes 44 SNPs, 3 gender marker, and 5 copy number controls (Agena Bioscience).

### Assay Protocol: Sample ID Panel (Master Mix Reagents)

### 1. Performing PCR Amplification

Genomic DNA from the samples were amplified using the supplied PCR primers in either a 96-well or a 384-well plate in a final reaction volume of 5 μl. The Sample ID Panel required 2 μl of DNA sample per well.
a. The PCR cocktail was prepared in a 1.5 mL microcentrifuge tube placed on ice or a cold block by adding reagents in the order and quantities in which they are listed in Table 2 below.

TABLE 2
PCR Cocktail (Master Mix Reagents)

| Reagent | Per Reaction (μL) |
|---|---|
| HPLC-grade water | 0.66 |
| IPEX Pro Master Mix, PCR Mix | 0.84 |
| PCR Primer | 1.0 |
| Q-Mix | 0.5 |
| PCR Cocktail Final Volume | 3.0 |
| DNA | 2.0 |
| PCR Reaction Final Volume | 5.0 |

b. The tube was vortexed 5 times and briefly centrifuged.
c. 3 μL of PCR cocktail was dispensed into wells of a new microtiter plate.
d. 2 μL of sample DNA was dispensed to each well, for a final PCR reaction volume of 5 μL.
e. The PCR reaction plate was sealed, pulse vortexed 5 times, then centrifuged at 1000×g for 15 seconds.
f. Individual wells were visually inspected from the bottom of the PCR reaction plate to confirm uniform and adequate cocktail solution was present in every well before continuing.
g. Thermocycling was performed using the following conditions:
   i. 95° C. 2 minutes
   ii. 95° C. 30 seconds
   iii. 56° C. 30 seconds
   iv 72° C. 1 minute
   v 10° C. Hold
Steps ii, iii and iv were repeated for 45 cycles.

### 2. Performing the SAP Treatment

a. The SAP cocktail was prepared in a 1.5 mL microcentrifuge tube on ice by adding reagents in the order and quantities in which they are listed in Table 3 below.

TABLE 3
SAP Cocktail (Master Mix Reagents)

| Reagent | Per Reaction (μL) |
|---|---|
| HPLC-grade water | 1.7 |
| IPLEX Pro Master Mix, SAP Mix | 0.3 |
| SAP Cocktail Final Volume | 2.0 |

b. The tube was vortexed 5 times and briefly centrifuged.
c. The reaction plate was centrifuged at 1000×g for 15 seconds.
d. 2 μL of SAP cocktail was dispensed to each well of the reaction plate.
e. The reaction plate was sealed, pulse vortexed 5 times, then centrifuged at 1000×g for 15 seconds.
f. Individual wells were visually inspected from the bottom of the reaction plate to confirm uniform and adequate cocktail solution was present in every well before continuing.
g. Thermocycling was performed using the following conditions for 1 cycle:
   37° C. 40 minutes
   85° C. 5 minutes
   10° C. Hold

### 3. Performing the IPLEX Pro Extension Reaction

a. The iPLEX Pro extension cocktail was prepared in a 1.5 mL microcentrifuge tube placed on ice by adding reagents in the order and quantities in which they are listed in Table 4 below.

TABLE 4
IPLEX Pro Extension Reaction Cocktail (Master Mix Reagents)

| Reagent | Per Reaction (μL) |
|---|---|
| HPLC-grade water | 1.0 |
| IPLEX Pro Master Mix, SBE Mix | 0.2 |
| Extend Primer | 0.8 |
| Extension Reaction Cocktail Final | 2.0 |
| Volume | |

b. The tube was pulse vortexed 5 times and briefly centrifuged.
c. The SAP-treated reaction plate was centrifuged at 1000×g for 15 seconds.
d. 2 L of extension reaction cocktail was dispensed into each well of the reaction plate. Tips were changed after each dispense.
e. The reaction plate was sealed, pulse vortexed 5 times, then centrifuged at 1000×g for 15 seconds.
f. Individual wells were visually inspected from the bottom of the reaction plates to confirm uniform and adequate solution was present in every well before continuing.
g. The reaction plate was thermocycled using the following reaction conditions:

| | | |
|---|---|---|
| i. | 95° C. | 30 seconds |
| ii. | 95° C. | 5 seconds |
| iii. | 52° C. | 5 seconds |
| iv. | 80° C. | 5 seconds |
| v. | 72° C. | 3 minutes |
| vi. | 10° C. | Hold |

Steps ii, iii, and iv were repeated for 40 cycles. Steps iii and iv were repeated for 5 cycles within the 40 cycles.

### 4. Water Addition

a. HPLC-grade water was added to each well of the reaction plate using a 12-channel multipipettor.
   i. For 96-well plates, 41 μL was added.
   ii. For 384-well plates, 16 μL was added.
b. The plate was sealed and centrifuged at 1000×g for 1 minute.
c. The plate was processed on the MassARRAY System, using instrument setting for iPLEX Pro genotyping panels, and Genotype+Area for the process method in ChipLinker.

### 5. Data Analysis

The software in the MassArray system compares the tested DNA with a data base from previously tested samples and determines whether there is a match or not and reports it.

What is claimed is:

1. A method of analyzing and authenticating a sample from a subject comprising:
providing a blood volume of a blood sample from the subject contained within an absorbent probe of a biological fluid sampling device;
forming an extracted blood sample by contacting the absorbent probe with an extraction volume of an extraction solution;
forming a liquid chromatography blood sample by contacting the extracted blood sample with a liquid chromatography volume of a liquid chromatography blood sample solution;
provided that a drug or metabolite analyte is present in the liquid chromatography blood sample, detecting the drug or metabolite analyte by performing liquid chromatography on the liquid chromatography blood sample and then mass spectroscopy;
providing a body sample from the subject;
forming purified DNA by purifying the body sample;
forming a sample DNA fingerprint by performing a polymerase chain reaction (PCR) on the purified sample DNA; and
authenticating the blood sample from the subject by comparing the sample DNA fingerprint to a reference DNA fingerprint of the subject, wherein the sample DNA fingerprint and the reference DNA fingerprint comprise genomic DNA markers unique to the subject.

2. The method of claim 1, wherein the body sample comprises a portion of the extracted blood sample, a blood sample, a serum sample, a buccal swab sample, a saliva sample, a urine sample, a hair follicle sample, a tissue sample, or a combination thereof; or
wherein the subject is a patient, a criminal suspect, a drug test subject, an athlete, a sports player, or an employee.

3. The method of claim 2, wherein the blood sample has a volume of from about 20 μl to about 30 μl, the buccal swab sample has a volume of from about 100 μl to about 300 μl, the urine sample has a volume of from about 100 μl to about 300 μl; or wherein the purified sample DNA comprises from about 5 ng to about 100 ng of genomic DNA.

4. The method of claim 1, further comprising:
obtaining a reference body sample from the subject, wherein the reference body sample comprises a blood sample, a buccal swab sample, or a urine sample;
purifying reference DNA from the baseline identification sample; and
forming the reference DNA fingerprint of the subject by performing a PCR reaction on the purified reference DNA.

5. The method of claim 1, further comprising reporting a genetic match or a genetic mismatch between the sample DNA fingerprint and the reference DNA fingerprint of the subject; or wherein the reference DNA fingerprint comprises a database reference genetic profile; or wherein the genomic DNA markers comprise single nucleotide polymorphisms (SNPs), gender markers, or a combination thereof.

6. The method of claim 5, wherein the genomic DNA markers comprise from about 10 to about 50 SNPs or from 1 to about 5 gender markers, or a combination thereof.

7. The method of claim 1, further comprising analyzing the sample DNA fingerprint by performing liquid chromatography on the purified sample DNA and then mass spectroscopy; or wherein purifying sample DNA from the body sample comprises vacuum concentration of the sample DNA; or wherein purifying sample DNA from the body sample comprises:
provided the body sample comprises cells,
performing a cell lysis on the body sample to form a cell lysate; and
performing an alcohol extraction on the cell lysate.

8. The method of claim 1, wherein the biological fluid sampling device includes an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body, and
wherein the absorbent probe includes an absorbent material.

9. The method of claim 8, wherein the absorbent material includes a polyolefin, polyester, polyethylene, a porous carbonized material, or a combination thereof; or the absorbent probe includes an anti-coagulant.

10. The method of claim 1, wherein the blood volume is from about 5 μl to about 50 μl; or wherein the purified sample DNA has a volume of from about 2.5 μl to about 15 μl in the PCR reaction; or wherein the PCR reaction has a total volume of from about 5 μl to about 20 μl; or wherein the PCR reaction comprises from about 35 thermocycles to about 50 thermocycles.

11. The method of claim 1, wherein the extraction volume is from about 50 microliters to about 200 microliters; or
the extraction solution includes a ratio of from about 4:1 to about 9:1 of an organic polar solvent to water; or
the extraction solution is an aqueous solution that includes from about 70% to about 90% volume percent of an organic polar solvent based on a total volume of the extraction solution.

12. The method of claim 11, wherein the organic polar solvent is selected from the group consisting of methanol, ethanol, diethylene glycol, glycerin, acetic acid, and 2-aminoethanol.

13. The method of claim 1, further comprising, adding an internal standard volume of an internal standard solution before or during forming the extracted blood sample; or
adding an internal standard volume of an internal standard solution before or during forming the liquid chromatography blood sample; or
wherein the PCR reaction comprises an internal DNA quality control, an internal DNA quantity control, or a combination thereof.

14. The method of claim 13, wherein the internal standard volume is from about 5 microliters to about 20 microliters.

15. The method of claim 1, further comprising, forming the liquid chromatography blood sample by centrifuging the liquid chromatography blood sample solution for a centrifuge duration at a centrifuge rate, and then separating the liquid chromatography blood sample from any solids formed during centrifugation.

16. The method of claim 1, wherein the liquid chromatography blood sample solution includes an aqueous solution of from about 1:8 to about 1:2 methanol to water, and from about 0.01% to about 2% formic acid based on a total volume of the liquid chromatography blood sample solution.

17. The method of claim 1, further comprising, forming the extracted blood sample by vortexing the absorbent probe and the extraction solution for an extraction vortex duration.

18. The method of claim 1, further comprising, forming the liquid chromatography blood sample by removing from about 80% to 100% of a liquid from the extracted blood sample to form an extracted blood sample residue, and vortexing the extracted blood sample residue in contact with the liquid chromatography volume of the liquid chromatography blood sample solution for a residue vortex duration.

19. The method of claim 1, further comprising, performing liquid chromatography by pumping a first mobile phase and a second mobile phase through a solid phase column at a pressure of from about 5,000 kPa to about 35,000 kPa at a rate of from about 0.1 ml per minute to about 2 ml per minute, wherein the solid phase includes biphenyl, the first mobile phase includes from about 0.03% to about 1% formic acid and from about 0.03% to about 1% ammonium formate in water, and the second mobile phase includes from about 0.03% to about 1% formic acid in methanol.

20. The method of claim 19, further comprising, provided that a drug or metabolite analyte is present in the liquid chromatography blood sample, quantifying an amount of the drug or metabolite analyte in the liquid chromatography blood sample by comparing an amount of the drug or metabolite analyte detected relative to an amount of the drug or metabolite analyte in the internal standard solution.

21. A method of analyzing and authenticating a blood sample comprising:
providing a blood volume of a blood sample contained within an absorbent probe of a biological fluid sampling device, wherein the biological fluid sampling device comprises an elongated and tapered body extending along a longitudinal axis and having a smaller diameter first end and a larger diameter second end, the second end forming a conical internal recess, wherein the conical internal recess extends along a length of the longitudinal axis, connecting the second end to the absorbent probe at the first end of the body;
forming an extracted blood sample by contacting the absorbent probe with an extraction volume of an extraction solution and an internal standard volume of an internal standard solution;
forming the liquid chromatography blood sample by removing from about 80% to 100% of a liquid from the extracted blood sample to form an extracted blood sample residue, mixing the extracted blood sample residue with a liquid chromatography volume of a liquid chromatography blood sample solution, and centrifuging the liquid chromatography blood sample solution for a centrifuge duration and a centrifuge rate, and then separating the liquid chromatography blood sample from any solids formed during centrifugation;
provided that a drug or metabolite analyte is present in the liquid chromatography blood sample, quantifying an amount of the drug or metabolite analyte by performing liquid chromatography on the liquid chromatography blood sample and then mass spectroscopy;
providing a body sample from the subject;
forming purified DNA by purifying the body sample;
forming a sample DNA fingerprint by performing a polymerase chain reaction (PCR) on the purified sample DNA;
comparing the sample DNA fingerprint to a reference DNA fingerprint of the subject, wherein the sample DNA fingerprint and the reference DNA fingerprint comprise genomic DNA markers unique to the subject; and
determining a genetic match or a genetic mismatch between the sample DNA fingerprint and the reference DNA fingerprint of the subject based on the comparison.
